(19) United States
(12) Patent Application Publication
(10) Pub. No.: US 2022/0331488 A1
(43) Pub. Date: Oct. 20, 2022

(54) Prosthetic Tissue Treatment For Desirable Mechanical Properties

(71) Applicant: TissX, Inc., Plymouth, MN (US)

(72) Inventors: Matthew F. Ogle, Edina, MN (US); William Robert Holmberg, Hudson, WI (US)

(21) Appl. No.: 17/375,639

(22) Filed: Jul. 14, 2021

Related U.S. Application Data
(60) Provisional application No. 63/176,329, filed on Apr. 18, 2021.

(51) Int. Cl. A61L 27/36; B33Y 80/00; B33Y 50/00; B29C 64/386

(52) U.S. Cl. CPC A61L 27/3687; A61L 27/3625; A61L 27/3691; B33Y 80/00; B33Y 50/00; B29C 64/386; B29L 2031/753

(57) ABSTRACT

Tissue processing techniques are described involving association of tissue with a form under pressure to more precisely adapt the crosslinked tissue to the form. Pressure can be applied through holding of the issue on a porous form with suction on the form maintain tight adherence of the tissue on the form. In some embodiments, the tissue on the form is placed with the crosslinking solution in a bag that is then vacuum sealed to have the evacuated bag hold the tissue on the form. Whether or not the tissue is crosslinked on a form, glutaraldehyde can be used for crosslinking in a substantially unpolymerized state to achieve distinct crosslinked tissue properties.

### CROSS REFERENCE TO RELATED APPLICATIONS

This application claims priority to copending U.S. provisional patent application 63/176,329 filed Apr. 18, 2021, to Ogle et al., entitled “Tissue Treatment,” incorporated herein by reference.

### FIELD OF THE INVENTION

The invention relates to the treatment of tissue portions of medical devices such as bioprosthetic devices, with selected, treatment solutions to result in a specified medical article, which is packaged and sterile. The tissue treatment can comprise one or more of procurement, selection methods, sanitation, bioburden reduction, fixation, formation, storage, terminal sterilization, and packaging. In particular, tissue fixation may be performed in contact with a form or mold to provide for more precise prosthesis structure, and fixation can be performed under conditions to better maintain tissue properties.

### BACKGROUND

A variety of medical devices such as prostheses can be used to repair or replace damaged or diseased organs, tissues and other structures in humans and animals. Some of these medical devices incorporate tissue or synthetic material as at least a component of the prosthesis. Prostheses generally should be biocompatible due to possible prolonged contact with bodily fluids.

It is often necessary or desirable to treat the natural or synthetic tissue prior to use to improve performance of the prosthetic. Tissue used in prostheses typically is fixed prior to use. Fixation stabilizes the tissue, especially from enzymatic degradation, and reduces antigenicity.

In addition, a prosthesis can be treated with a variety of agents to reduce calcification, i.e., the deposit of calcium salts, particularly calcium phosphate (hydroxyapatite), following implantation in a recipient. Calcification affects the performance and structural integrity of medical devices constructed from these tissues, especially over extended periods of time. For example, calcification is the primary cause of clinical failure of bioprosthetic heart valves. The tissue can also be decellularized in an effort to reduce the effects of calcification and to promote cell growth. Other possible treatments include the application of, for example, antimicrobials, antioxidants and antithrombotic. Some treatments for medical devices involve the use of potent chemicals, which can result in degradation of delicate portions of the medical device.

### SUMMARY OF THE INVENTION

In a first aspect, the invention pertains to a method for crosslinking a tissue comprising crosslinking tissue with a dialdehyde crosslinking agent under a low oxygen atmosphere, wherein the dialdehyde crosslinking agent is provided in a substantially unpolymerized state.

In a further aspect, the invention pertains to a method for forming structures comprising fixed tissue, the method comprising the steps of: placing a portion of tissue on a form with a contoured shape; supporting the tissue on the form using pressure differentials; and crosslinking the tissue supported on the form using a crosslinking solution.

In another aspect, the invention pertains to a method for forming structures comprising fixed tissue, the method comprising the steps of: supporting a portion of tissue on a form with a contoured shape, wherein the form is porous; and crosslinking the tissue in contact with the form using a crosslinking solution.

In an additional aspect, the invention pertains to a method of making a form useful for crosslinking tissue for a bioprosthesis intended for a specific patient, the method comprising the steps of: imaging a defective tissue section in the specific patient; adjusting the image to correct the defect and to form an image of a corrected tissue section; and performing three dimensional printing of the image of the corrected tissue section.

### BRIEF DESCRIPTION OF THE DRAWINGS

FIG. 1 is a flow diagram of the general process over the whole cycle of the tissue.

FIG. 2 is a perspective view of a form in the shape of an aortic heart valve.

FIG. 3 is a perspective view of a form in the shape of a section of blood vessel with two branched vessels.

FIG. 4 is a schematic perspective view of an aortic heart valve form in contact with stitched together tissue portions sealed in an evacuated bag with crosslinking solution.

FIG. 5 is a schematic perspective view of a porous aortic heart valve form with associated tissue held onto the form with suction applied with a pump, in which the form and tissue are immersed in crosslinking solution and any liquid removed by the pump is recirculated into to the crosslinking bath.

### DETAILED DESCRIPTION OF THE INVENTION

Improved tissue processing is provided through one or more process improvement, in particular crosslinking with glutaraldehyde having fewer glutaraldehyde polymers and/or crosslinking tissue in contact with a formed surface to give the crosslinked tissue a natural curved shape. Processing of the crosslinked tissue can further comprise treatments to improve biocompatibility and/or reduce calcification. Due to self-polymerization by glutaraldehyde, crosslinking with glutaraldehyde can provide different properties due to varying degrees of glutaraldehyde self-polymerization. Through the reduction or elimination of glutaraldehyde polymerization, significantly improved crosslinking can be achieved with proper preparation and processing with the glutaraldehyde with less or no polymerization. Conventional tissue processing involves crosslinking, i.e., fixing, the tissue and then forming the tissue into a desired prosthetic device. The tissue can be cut directly based on the form to have a carefully constructed tissue structure. In some embodiments, the form can be made based on imaging of the patient's anatomy with potential corrections for defects such that a custom prosthesis can be made using the imaged generated form. The form can be made, for example, using 3-D printing.

In some embodiments, the treatments of tissue for medical devices involve formation of geometries, which match complex anatomy of the natural structures that are being repaired or replaced. Thus, advantages can be obtained in fixing and forming tissue for such devices contoured in those shapes. In particular, the natural, unstressed shape of the product then can correspond to the shape of the natural anatomy of a specific patient. This design to match the natural anatomy can improve function. In a few circumstances with naturally shaped prosthetic tissue available as a hard tissue, the processed tissue can maintain the shape of the harvested tissue. For example, meniscus implants for humans can be formed from meniscus tissue obtained from non-human sources, such as bovine or porcine sources, for formation into the prostheses substantially maintaining the initial shape. See, for example, published U.S. patent application 2020/0038193 to Li et al., entitled “Collagen-Based Meniscus Implants,” incorporated herein by reference. However, soft tissues are of particular interest even though some of the crosslinking processing is more generally applicable, and for soft tissue shaping the tissue during crosslinking can be advantageous.

Tissues can be fixed by crosslinking. Fixation provides mechanical stabilization, for example, by preventing enzymatic degradation of the tissue and reduces the antigenicity. Glutaraldehyde, formaldehyde or a combination thereof is typically used for fixation, but other fixatives can be used, such as epoxides and other difunctional aldehydes. Aldehyde functional groups are highly reactive with amine groups in proteins, such as collagen.

In some embodiments, treatments for fixed tissue within prosthetic medical device can address negative consequences of the fixation process, such as cytotoxicity and/or calcification. Aldehyde crosslinking tends to make the tissue cytotoxic. This cytotoxicity appears to be due to unreacted aldehyde functional groups. While processing approaches to reduce calcification may reduce the level of cytotoxicity, residual cytotoxicity can remain a problem with respect to inhibiting colonization of the crosslinked tissue by mammalian cells both in vitro in a cell culture and in vivo in a patient following implantation. Treatment of tissue to remove cytotoxicity is described in published U.S. patent application 2003/0130746 to Ashworth et al. (hereinafter the '746 application), entitled “Biocompatible Prosthetic Tissue,” incorporated herein by reference. Calcification, i.e., the deposit of calcium salts especially calcium phosphate (hydroxyapatite), occurs in and on some materials used in the production of implantable bioprostheses, i.e., bioprosthetic devices. This calcification can affect the performance and structural integrity of medical devices constructed from these biomaterials, especially over extended periods of time. For example, calcification is the primary cause of clinical failure of bioprosthetic heart valves made from porcine aortic valves or bovine pericardium. Calcification also can significantly affect the performance of bioprostheses constructed from synthetic materials, such as polyurethane. The use of metal ions to reduce calcification is described in U.S. Pat. No. 6,302,909 to Ogle et al., entitled “Calcification-Resistant Biomaterials,” incorporated herein by reference.

Some treatments for medical devices help with preservation or storage, such as dehydration, for dry storage, and afford alternative sterilization methods.

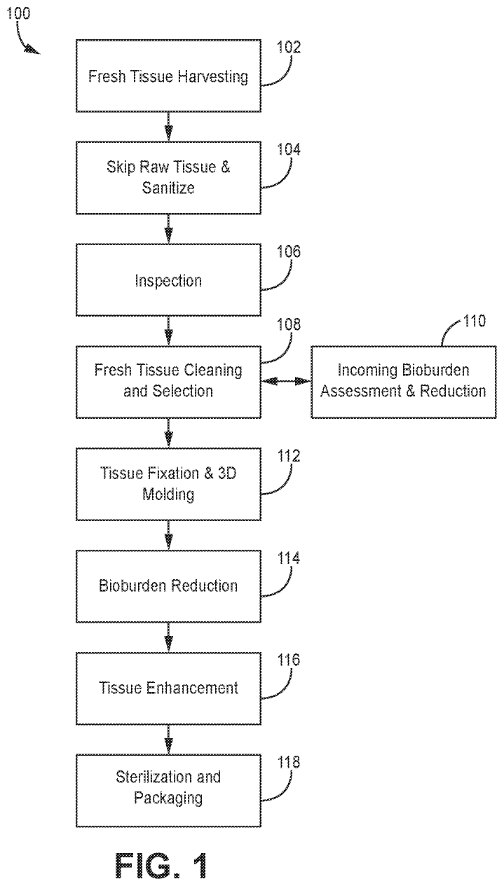

Referring to FIG. 1, the processing 100 can feature the follow steps including: Fresh Tissue Harvesting 102, Ship Raw Tissue and Sanitize 104, Inspection 106, Fresh Tissue Cleaning and Selection 108 (which further can include Incoming Bioburden Assessment and Reduction 110), Tissue Fixation and 3D Molding 112, Bioburden Reduction 114, Tissue Enhancement 116, and Sterilization/Packaging 118. These steps are just one example and are not limiting. In some embodiments, additional, fewer, and/or alternative steps can be used. While improvements are focused on the fixation step, the various steps can interrelate. In the following, these steps are organized in the following sections: Procurement, Selection, Sanitation, Formation, Fixation, Tissue Enhancement, and Storage/Sterilization/Packaging. The improvements described herein can be advantageous for certain applications of the prosthetic tissue. In particular, there is interest in prosthetic heart valves and other cardiovascular structures.

### Procurement:

Appropriate materials for tissue-based medical article can be formed from natural materials, synthetic protein tissue matrices and combinations thereof. “Bioprosthesis” is used in a broad sense to include prosthetic devices comprised of a natural material component that is joined together with other natural or synthetic materials. Synthetic tissue matrices can be formed from extracellular matrix proteins that are crosslinked to form a tissue matrix. Extracellular matrix proteins are commercially available. For example, medical grade collagen from extracellular matrix is commercially available form Collagen Solutions Plc., UK. Natural, i.e. biological, material for use in the prosthetic materials described herein includes, for example, reconstituted tissue from ECM or relatively intact tissue as well as decellularized tissue. Extracellular matrix generally comprises collagen, elastin, glycoproteins, and other structural proteins and possibly polysaccharides. Natural tissue materials may be obtained from, for example, native heart valves, portions of native heart valves such as roots, walls and leaflets, pericardial tissues such as pericardial patches, connective tissues, bypass grafts, tendons, ligaments, skin patches, blood vessels, cartilage, dura mater, skin, bone, fascia, submucosa, umbilical tissues, peritoneal tissues and the like.

Natural tissues are derived from a various animal species, typically mammalian, such as human, bovine, porcine, canine, seal or kangaroo. These tissues may include the whole organ, including, for example, homografts and autografts. These natural tissues generally comprise collagen-containing material. Natural tissue is typically, but not necessarily, soft tissue. Tissue materials are particularly useful for the formation of tissue heart valve prostheses.

After examining tissue for potential damage from harvesting that can make the tissue unsuitable for use, then the tissue can be trimmed of any extraneous components, such as fat tissue, membranes, or other undesirable components. So pericardial tissue after trimming can be a relatively smooth essentially flat sheet of tissue.

The tissue can be decellularized. Decellularization of tissue is described, for example, in published U.S. patent application 2020/0237961 to Fernandez et al., entitled “Decellularized Biomaterial and Method for Formation,” incorporated herein by reference.

### Selection:

Selection processes can be facilitated with various measurement aids. For most applications starting with pericardial tissue, it is desirable for the tissue to be relatively uniform across the tissue. To evaluate the uniformity, light transmission, thickness, composition, 3d mapping using IR spectroscopy, atomic force microscopy (AFM), mechanical properties or a combination thereof may be used to select and characterize tissue specimens. Mechanical properties can involve measurements such as flection or other mechanical measures. Infrared 3-D scanners, AFM equipment and other relevant measurement equipment are commercially available.

One area or example is to determine orientation of collagen fiber in harvested pericardium tissue. This orientation can be used to guide crosslinking on a form structure to obtain desired physical properties of the fixed tissue. In pericardial tissue from an animal source, the collagen fibers would generally be oriented. The tissue will have mechanical properties corresponding with a less flexible tissue perpendicular to the fiber axis versus bending along an axis parallel to the fiber axis. Collagen orientation can be evaluated using light scattering and other non-destructive optical evaluation techniques. See, for example, Cochran R P, Kunzelman K S, Chuong C J, et al. Nondestructive analysis of mitral valve collagen. ASAIO Trans 1991; 37: M447-M448, incorporated herein by reference.

### Sanitation/Cleaning:

Rinsing of tissue in a simple saline or buffer allows for reduction of an unspecified range of microorganisms. Under these non-specific conditions, the characteristics of the microorganism population can be reduced initially in the tissue, e.g., elimination of nature of contamination of the tissue upon receipt. Additional rinsing and/or soaking of tissue in specialized solutions can be used to further reduce organisms. For example, solutions can include: Sodium azide, isopropyl alcohol (IPA), other alcohols, and/or detergents. Additional solutions can be used to decellularize the tissue, although the cleaning protocols themselves provide some decellularization.

Decellularization can be performed to avoid significant damage to the extracellular matrix, such as through the incorporation of protease inhibitors. High concentration of surfactants and adjustment of the ionic strength can be used to help effectuate decellularization along with physical agitation and/or ultrasonic treatment.

### Formation

The invention contemplates using forms or molds to create structures to correct diseased anatomy. For example, we could mold an anatomical structure, such as a heart valve leaflet, and apply the tissue over the resulting form. With the tissue in contact with the form, the tissue can be crosslinked to impose the molded shape as a neutral (unstressed) tissue structure. In some embodiments, a general shape can be formed, optionally in a set of sizes to be selected for a patient, and the prostheses formed accordingly.

In additional or alternative embodiments, imaging (for example, CT, x-ray, ultrasound, MRI, or other imaging technique) can be used to get an image of a particular patient's defective organ, vessel or other tissue. The image can be used to make a three dimensional (3D) virtual model. Images can be manipulated in DICOM standard imaging format. Commercial image manipulation software includes, for example, OsiriX-DIACOM (Pixmio, Switzerland) and SYNOPSIS® (CA, USA). Imaging systems can interface with 3D printers. The 3D virtual model can be revised or corrected to make a revised 3D virtual model to repair the defect. The revised virtual model can be input into a 3D printer to make a custom tailored form corresponding to the patient's organ, vessel or other structure, which can be corrected of the defect in the image prior to performing the 3D printing. The custom form can then be used to contour tissue to make a custom prosthesis in a similar way as a standard form is used to crosslink the tissue. 3D printers are readily available commercially for forming this type of form.

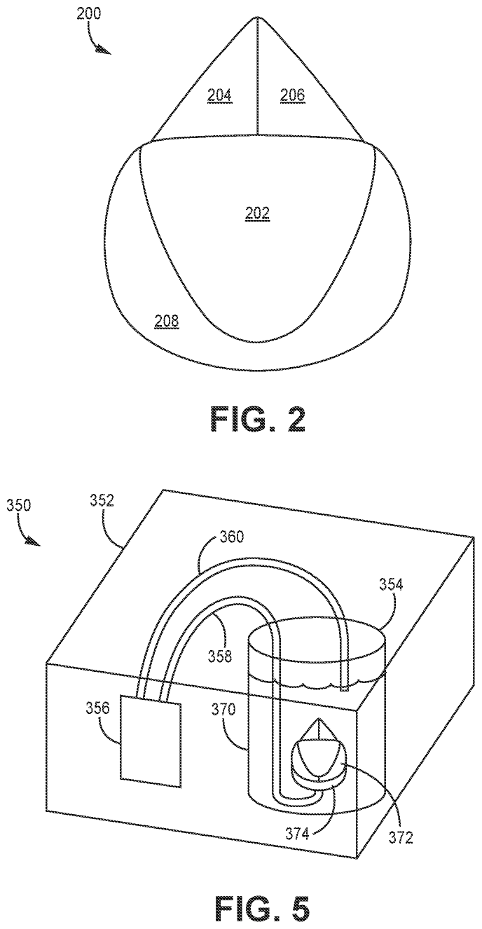

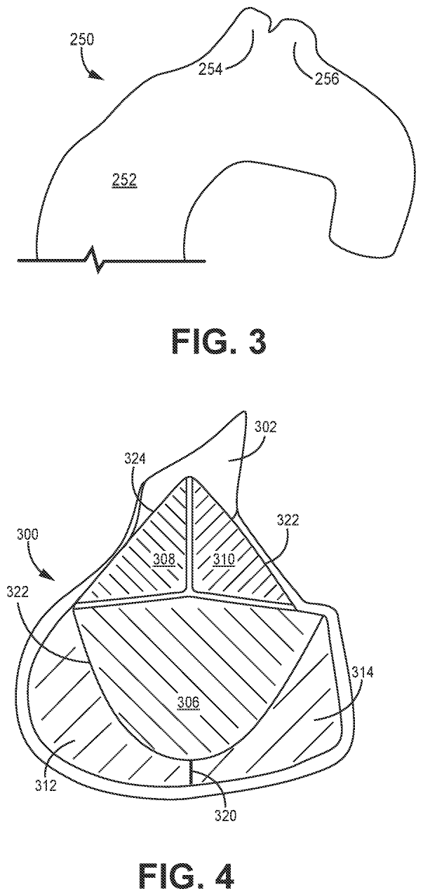

FIG. 2 depicts a form for leaflets for an aortic heart valve. Heart valve form 200 has a shape of an aortic heart valve. Heart valve form 200 comprises curved surfaces for three leaflets 202, 204, 206 as well as a base corresponding to the root 208. FIG. 3 depicts a form 250 for a section of a blood vessel 252 with two sections of branched vessels 254, 256. Defects can be corrected prior to making the form, such as using imaging software associated with 3D printing, and/or using physical adjustment of the form after the initial form is made. Other appropriate forms can be similarly generated.

### Molding/Process Overview for a Particular Embodiment:

Current medical imaging can be used to create computational 3D digital model, these models can be revised or modified.
The form can be reviewed in 3D software along with fixing to revise the structure or defective diseased organ or vascular structure, by the physician or technician.
The from can then be printed to scale with a 3D printer, such as printed on a 3D polymer printer, to create a form of the objective structure or shape to for use for crosslinking tissue. (see FIGS. 2 and 3)
Making the tissue structure over the form/mold. This involves linking the form to the tissue. By laying tissue over the form to create the contour. Tissue sections can be sewn with suture or the like or otherwise fastened together to form a tissue structure to be crosslinked on the mold/form.
Then, in some embodiments, the tissue over the form/mold are placed in a vacuum bag.
The bag is then filled with an appropriate volume of fixation solution (like Glut)
The air and excess solution are removed from the bag with a vacuum, this presses the tissue to the form/mold. Then, the bag can be sealed.
The tissue is allowed to fix for appropriate period of time.
Removing the fixed tissue from the mold is the final step.

Other: methods may include a porous mold when communication with the mold is created with the holes in the mold formed mechanically or by forming a porous polymer material.

Other example may include using balloons to hold tissue in a ring structure. For example, a cylindrically shaped balloon can be used to form a section of cylindrical tissue portion, which can be used to replace a section of a blood vessel or similar tubular shaped bodily element. After crosslinking, the balloon can be deflated to separate them from the tissue. Other balloon shapes can be similarly used. Such as shapes corresponding to FIGS. 2 and 3, which can then correspondingly correspond to balloons.

In another example, generalized vessel shapes and sizes could be created from 3D images and printed when needed.

A combination of any of the above methods may also be employed.

### Fixation

A reference to fixation historically refers to a process to preserve tissue, although the boundaries referred to can cover a range of processing. In the context of bioprostheses, fixation generally refers to a crosslinking process in which extracellular matrix proteins and perhaps other biological compositions are crosslinked to each other. Crosslinking can provide desirable effects and can, for example, sterilize the tissue, mechanically stabilize the extracellular matrix, remove antigens, and inactivate digestive enzymes that can digest the tissue. A variety of bioprostheses incorporate tissue as at least a component of the prosthesis. Such bioprostheses are used to repair or replace damaged or diseased organs, tissues and other structures in humans and animals. Tissue used in bioprostheses typically is chemically modified or fixed prior to use. Bioprostheses generally are designed also to be biocompatible due to prolonged contact with bodily fluids and/or tissues. Biocompatibility suggests minimally sufficient lack of toxicity and avoidance of extreme immunological response.

Typical fixation agents act by chemically crosslinking portions of the tissue, especially collagen fibers. Crosslinking compounds include, for example, dialdehydes such as butanedial (succinaldehyde), hexanedial and glutaraldehyde, carbodiimides, epoxies and oxidative fixation compounds such as photoxidative fixation agents. Glutaraldehyde particularly has found widespread use in part because it can be used for crosslinking at an approximately physiological pH under aqueous conditions. In addition to crosslinking the tissue, glutaraldehyde sterilizes the tissue and reduces the antigenicity of the tissue by the recipient. Glutaraldehyde has generally been effective to yield reasonable mechanical properties for the fixed tissue.

While appropriate fixation of the tissue is needed to use xerograft tissue that would otherwise elicit strong tissue rejection, fixation generally is associated with decreased flexibility of the tissue. Also, fixation with glutaraldehyde has been associated with calcification, i.e., the deposit of calcium salts, especially calcium phosphate (hydroxyapatite), following implantation in a recipient. This is probably due to cytotoxicity resulting in necrosis of tissue leading to calcification. Calcification affects the performance and structural integrity of bioprosthetic devices constructed from these tissues, especially over extended periods of time. For example, calcification is the primary cause of clinical failure of bioprosthetic heart valves.

The chemical nature of the glutaraldehyde-amine reaction is complex due to the reactivity of the glutaraldehyde molecule as well as the self-polymerization of dialdehydes. The most important component of the reaction products of an aldehyde and a primary amine involves the formation of a Schiff s base wherein the nitrogen forms a double bond with the aldehyde carbon, replacing the double bond with the oxygen. However, a variety of complex structures can be formed with free aldehyde groups. The chemistry of glutaraldehyde crosslinking is described further in “Bioprosthesis derived crosslinked and chemically modified collagenous tissues,” chapter in Collagen, vol. III, Nimni et al., Eds. 1988, incorporated herein by reference.

Desirable crosslinking on a form can be conveniently performed with glutaraldehyde or other collagen crosslinking agent. Improved gluraraldehyde crosslinking is described in the following section. The use of carbodiimides (R₁—N═C═N—R₂) is described in published U.S. patent application 2002/0081564 to Levy et al., entitled “Stabilization of Implantable Bioprosthetic Devices,” incorporated herein by reference. The use of a combination of a diamine crosslinking agent, a carbodiimide or succinimide, and a coupling enhancer, such as butyl alcohol or N-hydroxysulfosuccinimide, is described in published U.S. patent application 2004/0253291 to Girardot et al., entitled “Calcification Resistant Fixation,” incorporated herein by reference. The use of polyepoxy amines is described in U.S. Pat. No. 6,391,538 to Vyavahare et al., entitled “Stabilization of Implantable Bipoprosthestic Tissue,” Incorporated herein by reference. The inhibition of elastin degradation using tannins in conjunction with glutaraldehyde crosslinking is described in Isenberg et al., “Tannic acid treatment enhances biostability and reduces calcification of glutaraldehyde fixed aortic wall,” Biomaterials 26 (2005) 1237-1245, incorporated herein by reference. Pentagalloyal glucose (PGG) is a tannic acid derivative that can be an effective replacement for tannic acid, and other tannins may also be used.

Desirable crosslinking on a form can be performed on a porous structure to facilitate wetting of the interior of the tissue in contact with the form. In general, porous forms can be made by molding a polymer with a soluble salt, soluble polymer, or other suitable pore forming agent. After making the form, the pore forming agent can be removed, such as dissolving the pore forming agent to make the porous article. Use of an appropriate amount and size of a pore forming agent, the porosity can be controlled. Generally, any pores should not be so large that they significantly mark the adjacent tissue during processing. In some embodiments, the average pore size can be from about 1 micron to 500 microns, although it is recognized that additional ranges of average ore sizes within this range are contemplated. Porous articles can also be formed by 3D printing. See, Mu et al., “Porous polymeric materials by 3D printing of photocurable resin,” Materials Horizons, Issue 3, 2017 (doi.org/10.1039/C7MH00084G), incorporated herein by reference.

The use of a porous form can provide the ability to more uniformly crosslink the tissue in a desirable timeframe since the crosslinking agent can in principle better wet the tissue on the surface of the form. In some embodiments, the tissue can be sutured together or otherwise secured to stabilize the tissue on the form for crosslinking. While the advantage from the porous form can be obtained through careful immersion of the tissue in contact with the form, the use of the porous form can be combined with other processing improvements.

For example, the tissue mounted on the form can be placed into a plastic bag with sufficient crosslinking agent, and then the bag can be sealed and evacuated with an appropriate negative pressure device. The bag seals around the tissue on the form with sufficient crosslinking liquid to achieve the desired crosslinking. Generally, the volume of fluid would range from about 0.5 to about 20 times the tissue volume, in further embodiments from about 0.75 to about 15 times the tissue volume, and in additional embodiments form about 1 to about 12 times the tissue volume.

Referring to FIG. 4, an example of crosslinking in a sealed evacuated bag is depicted for crosslinking system 300 for an aortic heart valve prosthesis. Crosslinking system 300 comprises sealed evacuated bag 302 holding tissue 304 overlying a form (under tissue) and crosslinking solution occupying space between the tissue and the bag as well as impregnating the tissue and the form if porous. In this embodiment, tissue 304 comprises tissue portions sutured together including leaflets 306, 308, 310 and wall portions 312, 314. Generally, another wall portion would be used, and this additional wall portion can be located at the hidden back of the structure shown in FIG. 4. Leaflets have edges that meet in a closed configuration and that separate as the valve opens in response to pressure changes, and the leaflets (cusps) should be appropriately flexible to open and close with the leaflets forming an appropriate seal. While various portions of tissue can be cut to form the heart valve prosthesis, as shown in FIG. 4, six portions of tissue are assembled with five of the sections being visible and the sixth section being equivalent to wall portions 312, 314. Four of six suture seams 320, 322, 324, 326 are visible in FIG. 4, and two hidden seals are equivalent to seam 320. Various other tissue sections can be sutured differently into the heart valve prosthesis, and the suggested suturing in FIG. 4 is just a representative example.

In additional or alternative embodiments, negative pressure can be applied at the bottom of the porous form to hold the tissue on the form with negative pressure. The suction supplying the negative pressure can be relative low pressure differential to avoid any damage to the tissue and to reduce any removal of crosslinking fluid. Crosslinking fluid can be circulated back into the tissue holding container to avoid loss of fluid from the pumping on the porous form. In general, the pressure can be selected to provide the desired adherence of the tissue on the form, and various commercial pumps can be suitable. Referring to FIG. 5, crosslinking system 350 can comprise enclosure 352 to provide a low oxygen atmosphere for crosslinking, crosslinking vessel 354 which can be a beaker or other convenient vessel, pump 356 which can be any convenient design such as a peristaltic pump, outflow tubing 358 to apply suction, and inflow tubing 360 to return crosslinking fluid removed by pump 356. Crosslinking vessel 354 holds crosslinking fluid 370 and the structure being crosslinked. While this crosslinking approach is suitable for various structures, FIG. 5 depicts an aortic heart valve prosthesis being crosslinked, as an example. Thus, in this embodiment, structure 372 being crosslinked can be equivalent to crosslinking system 300 without sealed evacuated bag 302 with a porous form, which is optional in system 300, and with the crosslinking solution within sealed evacuated bag 302 replace with crosslinking fluid 370. Outflow tubing 358 interfaces with structure 372 with a cover 374 that forms a seal along a surface of the form that is free of tissue such that negative pressure applied by outflow tubing 358 communicates through the porous form to hold tissue onto the form. Cover 374 can be made from a rubber or other elastomer to attach with a friction fit, although other designs can be used.

### Improved Glutaraldehyde Crosslinking

Glutaraldehyde crosslinks through formation of bonds with amine groups in proteins. Glutaraldehyde (C₅H₈O₂, OCH₂(CH₂)₃CH₂O) is also known to self-polymerize into longer molecules that also have aldehyde groups. The distance between aldehyde groups represents a crosslink distance, and the crosslink distance has been identified as influencing the properties of crosslinked tissue. Presumably, essentially all chemically accessible amine groups in the tissue are crosslinked in fully crosslinked tissue. The amount in weight of bound crosslinking agent in the crosslinked tissue would be expected to correlate with the degree of polymerization of the glutaraldehyde. In the processes described herein, process designs are applied to limit the self-polymerization of glutaraldehyde to a low level, and corresponding tissue properties may be correspondingly improved.

Crosslinking of tissue with glutaraldehyde having reduced degrees of self-polymerization is described in U.S. Pat. No. 5,958,669 to Ogle et al. (the '669 patent), entitled “Apparatus and Method for Crosslinking to Fix Tissue or Crosslink Molecules to Tissue,” incorporated herein by reference. In the '669 patent, tissue is crosslinked in a solution in which glutaraldehyde is provided through a size exclusion membrane to block access to highly polymerized glutaraldehyde. Various dialysis membranes are described in the '669 patent to essentially filter the glutaraldehyde. Examples were presented with 10,000 Dalton dialysis bags to hold the tissue, which roughly corresponds to exclusion of polymers greater than 100mers of glutaraldehyde. Measure of shrink temperatures confirmed that the tissue crosslinked with tissue in the dialysis bag was fully crosslinked, which visual evaluation determined that the tissue crosslinked in the dialysis membranes was white rather than yellowish and was notably more flexible than conventional glutaraldehyde crosslinked tis sue.

The spectroscopic characterization of glutaraldehyde polymers using 235-nanometer UV absorption is described by Jones et al., “Formation of 235-nanometer absorbing substance during glutaraldehyde fixation,” Journal of Histochem Cytochem September 1974 22(9):913-915, incorporated herein by reference. The complexity of aqueous solutions of glutaraldehyde is reviewed by Migneault et al., “Glutaraldehyde: behavior in aqueous solutions, reaction with proteins, and application to enzyme crosslinking,” BioTechniques: 37:790-802 (November 2004), incorporated herein by reference. While Migneault et al. present a more complex picture, the presentation is not inconsistent with the presentation of Jones et al., although the monomers in solution are likely water adducts as a cyclic hemiacetal. Migneault et al. discuss likely structures of the polymer forms.

Commercial glutaraldehyde in aqueous solution are available in highly purified forms, which are believed to be essentially monomeric. In particular, these are sold for histology application, where they are asserted to provide superior results. See, Electron Microscopy Sciences, EM Grade Distillation Purified, “free from polymers and other contaminants,” which are sold in sealed containers over dry nitrogen and refrigerated at 4° C. to −18° C. The containers have a supplier provided shelf life. This EM Grade glutaraldehyde is a suitable starting material, although if desired, in-house distillation can be performed or activated charcoal purification described in Jones et al. can be used to prepare purified glutaraldehyde.

In general, the concentration of glutaraldehyde can influence the crosslinking rate, which could also influence the properties of the crosslinked tissue. Having a faster crosslinking rate can be beneficial form the standpoint of completing the crosslinking prior to extensive polymerization of the glutaraldehyde. Thus, there can be a balancing of factors in selecting a glutaraldehyde concentration. For crosslinking, the concentration of the glutaraldehyde can be selected to be from about 0.05 weight percent (wt %) to about 25 wt %, in further embodiments from about 0.1 wt % to about 20 wt %, in some embodiments from about 0.2 wt % to about 22.5 wt %, and in other embodiments from about 0.25 wt % to about 15 wt %. A person of ordinary skill in the art will recognize that additional ranges of concentration within the explicit ranges above are contemplated and are within the present disclosure. Glutaraldehyde concentration can be set through the use of a stock solution at the desired concentration or by starting with a particular stock solution and dilution with appropriate buffer formed with purified water, such as deionized water. Suitable buffers are described below, and the pH can be further adjusted accordingly.

Depending on the solution conditions, the glutaraldehyde polymerization can be relatively slow. This is explored somewhat by Jones et al., but they tested polymerization under atmospheric oxygen. It is believed that isolation from atmospheric oxygen slows the polymerization. While cooling can slow glutaraldehyde polymerization, cooling can also slow the crosslinking reaction, so cooling may or may not be desirable during tissue crosslinking. Similarly, heating can speed the crosslinking times, but also speed the polymerization. Speeding the crosslinking time is also desirable from a process efficiency perspective since less process space and equipment can be involved in processing a certain quantity of tissue if crosslinking s faster. Generally, the temperature can be from about 1° C. to about 85° C., and particular cooled embodiments from about 4° C. to about 15° C. or from 6° C. to about 12° C., and in particular heated embodiments from about 40° C. to about 80° C. or from about 42° C. to about 70° C. A person of ordinary skill in the art will recognize that additional ranges within the explicit ranges above are contemplated and are within the present disclosure. Glutaraldehyde can be considered substantially polymerization free if the solution has a UV (ultraviolet) absorption spectrum without a peak at 230 nm.

In the crosslinking process described herein, the crosslinking can be performed in an inert atmosphere substantially free of oxygen, such as a nitrogen or argon atmosphere. Nitrogen and argon are commercially available essentially free of oxygen, but processing may tolerate some level of oxygen presence. As used herein, a low oxygen atmosphere refers to no more than an oxygen partial pressure of 10 torr and in some embodiments no more than 1 torr. In some embodiments, the atmosphere is free of measurable oxygen. The crosslinking solution can be buffered at near physiological pH, where glutaraldehyde crosslinking has historically been effective. While glutaraldehyde polymerization is slowed at more acidic pH<7, polymer crosslinking is believed to be reversible at lower pH values, so tissue crosslinking may be less effective. While various interactions of reaction conditions can be selected for particular processing, in some embodiments the pH is selected from about 6 to 8 pH units, in further embodiments from about 6.5 to about 7.7, in other embodiments from about 6.7 to about 7.6, and in further embodiments from about 6.9 to about 7.5 pH units. A person of ordinary skill in the art will recognize that additional ranges of low oxygen partial pressure and pH within the explicit ranges above are contemplated and are within the present disclosure. Suitable buffers include, for example, phosphate, borate, bicarbonate, citrate, cacadylate, tris(hydroxymethyl)aminomethane (TRIS), 4-(2-hydroxyethyl)-1-piperazineethanesulfonic acid (HEPES), and 3-(N-morpholino)propanesulfonic acid (MOPS). The pH can be further adjusted with addition of appropriate amount of a strong acid (for example, HCl or HNO₃) or a strong base (for example, NaOH, KOH or NH₄OH) based on the buffer system.

Various commercial chambers are available for processing under inert atmospheres. For example, glove-box style environmental chambers suitable for use with inert atmospheres are widely available, and process scale generally guides apparatus selection. See, Terra-Universal (CA, USA), Fisher Scientific, and many others. Some process chambers provide a degree of temperature control. Sealed glutaraldehyde containers from a supplier can be opened within the inert atmosphere, and similarly in-house purified samples can be similarly controlled to start the processing with substantially monomeric glutaraldehyde. Similarly, purified nitrogen tanks are readily available commercially and can be connected to the process chamber.

Bioprosthetic heart valves from natural materials were introduced in the early 1960's. Bioprosthetic heart valves typically are derived from pig aortic valves or are manufactured from other biological materials such as bovine pericardium or pericardium from other mammalian species. Pericardium tissue, such as bovine pericardium or porcine pericardium, can be convenient for shaping into desired products with considerable ability to specify size and modify shape. Xenograft heart valves, i.e., heart valves originating from a donor of a species different from the species of the recipient, are typically fixed with glutaraldehyde prior to implantation to reduce the possibility of immunological rejection. Glutaraldehyde and similar crosslinkers reacts to form covalent bonds with free functional groups in proteins, thereby chemically crosslinking nearby proteins.

With respect to crosslinking time, generally the tissue is crosslinked to completion, especially for xenograft tissue, to avoid undesirable immune responses. The crosslinking time can be checked empirically to evaluate when the tissue is completed with respect to crosslinking. Processing time may depend on glutaraldehyde concentration, pH, and temperature as well as the particular tissue properties. Crosslinked tissue can be evaluated by thermal stability evaluated by the shrink temperature, through susceptibility to enzyme degradation and/or by evaluating the mechanical properties. The shrink temperature evaluated using differential calorimetry for conventional crosslinked tissue is generally between about 82° C. and 88° C. Generally, crosslinking times can be from about 5 minutes to about 10 days, in further embodiments from about 25 minutes to about 9 days, and in other embodiments from about 30 minutes to about 8 days. A person of ordinary skill in the art will recognize that additional ranges within the specific crosslinking time ranges above are contemplated and are within the present disclosure.

Tissue crosslinking can be performed with tissue placed freely in the crosslinking solution. In other embodiments, the tissue can be associated with a form, as discussed in the previous section.

### Tissue Products

The processing described herein can be applied to various tissue based structures/products. For example, tissue patches can be sections of tissue that are essentially planar and can be used for various surgical repair application. Tissue-based heart valve prostheses are of particular interest. Suitable heart valves include, for example, aortic valves or mitral valves. Prosthetic heart valves can be formed from natural heart valves, such as porcine aortic valves, or assembled from other fixed natural tissue, such as bovine pericardium. For tissue heart valve prostheses with any appropriate source of tissue, crosslinking on a form can be desirable.

The importance of bioprosthetic animal heart valves as replacements for damaged or diseased human heart valves has resulted in a considerable amount of interest in the long term performance of these valves and, in particular, in the effects of calcification on these xeno-transplants. Calcification, i.e., the deposit of calcium salts, especially calcium phosphate (hydroxyapatite), can occur in and on some materials of a medical article while contacting the patient's bodily fluids or tissue. Calcification can affect the performance and structural integrity of medical articles constructed from these materials, especially over extended periods of time. For example, calcification is the primary cause of clinical failure of bioprosthetic heart valves made from porcine aortic valves or bovine pericardium. Calcification can be particularly severe at stress points, such as where suture passes through tissue.

Generally, bioprosthetic heart valves begin failing after about seven years following implantation, and few bioprosthetic valves remain functional after 20 years. Replacement of a degenerating valve prosthesis subjects the patient to additional surgical risk, especially in the elderly and in situations of emergency replacement. While failure of bioprostheses is a problem for patients of all ages, it is particularly pronounced in younger patients. Over fifty percent of bioprosthetic valve replacements in patients under the age of 15 fail in less than five years because of calcification. Other prostheses made from natural and/or synthetic materials may also display clinically significant calcification.

As a result, there is considerable interest in preventing the deposit of calcium on implanted biomaterials, especially heart valves. Research on the prevention of calcification has focused to a considerable extent on the pretreatment of the biomaterial prior to implantation. Detergents (e.g., sodium dodecyl sulfate), toluidine blue or diphosphonates have been used to pretreat tissues in an attempt to decrease calcification by reducing calcium nucleation. Within a relatively short time, these materials tend to wash out of the bioprosthetic material into the bodily fluids surrounding the implant, limiting their effectiveness. The use of reservoirs of +3 metal ions, such as Fe⁺³ or Al⁺³, has been proposed, see Ogle et al., U.S. Pat. No. 6,302,909, entitled “Calcification-Resistant Biomaterials,” incorporated herein by reference.

Other approaches to reducing calcification have employed a chemical process in which at least some of the reactive glutaraldehyde moieties remaining after crosslinking are inactivated. Still other approaches have included development of alternative fixation techniques because evidence suggests that the fixation process itself may contribute to calcification and the corresponding mechanical deterioration. Some examples of alternative crosslinkers are triglycidyl amine (TGA), Genipin, carbodiimide, (such as 1-ethyl-3-(3-dimethylaminopropyl) carbodiimide (EDC), and D-Ribose. The crosslinking agents can be used in conjunction or separately, for example Glut+EDC.

### Toxicity Reducing Agents and Treatment of Crosslinked Tissue

Toxicity reducing agents include, for example, inorganic sulfur-oxygen compounds, organic sulfate compounds, amines, ammonia/ammonium ions and surfactants. Many toxicity reducing agents act by forming a chemical adduct with the aldehyde functional groups. For example, sulfate compounds can undergo nucleophilic addition to the carbonyl (C═O) group of an aldehyde. In some embodiments, combinations of toxicity reducing agents are used to contact the tissue either sequentially or simultaneously. Suitable combinations of toxicity reducing agents can be used to effectively reduce aldehyde reactivity within aldehyde crosslinked tissue, which can also be useful to reduce calcification.

Suitable toxicity reducing agents can include, for example, inorganic sulfur-oxygen anions or corresponding compounds. Desirable combinations of toxicity reducing agents can include, for example, an amine and inorganic sulfur-oxygen ions. Combinations of toxicity reducing agents can further comprise a surfactant and/or ammonium ions. Furthermore, a plurality of inorganic sulfur-oxygen anions, e.g., sulfates (bisulfates) and thiosulfates, can be combined to treat the crosslinked tissue. These toxicity reducing agents can be used to effectively reduce or eliminate residual cytotoxicity of the crosslinked tissue and to lower measurable residual aldehyde reactivity. Additional toxicity reducing agents include organic substituted sulfates. Amines generally can undergo nucleophilic addition at aldehyde functional groups to form an adduct. Treatment for reduction of toxicity is described further in published U.S. patent application 2003/0130746 to Ashworth et al., entitled “Biocompatible Crosslinked Tissue,” incorporated herein by reference.

### Storage:

After crosslinking and any post crosslinking processing, the tissue can be stored either before or after processing into a prosthetic structure. For example, for heart valve prostheses, the structure can be completed with any additional stitching of the tissue, and possible association with stents, frames, sewing cuffs or any other desired structural components. In some embodiments, it may be appropriate to dehydrate the tissue the tissue for storage, such as through freeze drying, vacuum drying, contact with a desiccating liquid, such as glycerol, or a desiccating solid, such as silica gel. Freeze drying can be performed using commercial freeze drying equipment. In other embodiments, the prosthesis is stored in solution. For tissues stored in solutions, the solutions can be bacterial static and solutions, which are either easily rinsed out or tolerated upon implantation of the bioprosthetic device. Some such solutions are antiseptic, high concentrations of sugars, alcohols, high or low PH, and/or organic salts. Storage containers can be nitrogen purged to keep the chemicals from oxidizing. In some embodiments, organic solvents can be used that are not conducive to bacterial or cell grown. Appropriate biocompatible compounds include, for example, starch and sugar solution, propylene glycol, alcohol, salts, soluble polymers, hydrogels, and proteins. Concentrations may range from 2%-100% by weight. Heat and agitation may also be used to super saturate solutions.

With respect to dry storage of bioprosthetic tissues, the tissue can be readily sterilized using gases, such as ethylene oxide, or radiation, such as UV or gamma rays. Dried tissue can be rehydrated shortly before use, if desired.

### Packaging:

Employing a properly selected package for the medical device or tissue bioprosthetic is significant for many reasons, including the fact that it is the first interaction for the user (medical professional) even before they even touch the product. Accessing medical article in its package contents while preserving sterility is valuable because if improperly accessed, the product becomes unusable, unsterile. This integrity of a sterile barrier needs to be preserved over a number of years. This can be accomplished in a few ways.

A rigid Jar can be a good vessel, or a sealed bag. Bag or pouch dry storage of bioprosthetic devices is convenient storage for dried prosthetic tissue.

Bioprosthetic tissue is generally sterilized prior to storage or in the storage container.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. The use of the term “about” herein refers to expected uncertainties in the associated values as would be understood in the particular context by a person of ordinary skill in the art.

What is claimed is:

1. A method for crosslinking a tissue comprising:
crosslinking tissue with a dialdehyde crosslinking agent under a low oxygen atmosphere, wherein the dialdehyde crosslinking agent is provided in a substantially unpolymerized state.

2. The method of claim 1 wherein the dialdehyde comprises glutaraldehyde.

3. The method of claim 2 wherein glutaraldehyde is in aqueous solution at a concentration from about 0.5 wt % to about 25 wt %.

4. The method of claim 1 wherein the crosslinking is performed under a nitrogen atmosphere.

5. The method of claim 1 wherein the crosslinking is performed at a pH from about 6.5 to about 7.7 pH units in a buffered solution.

6. The method of claim 1 wherein the crosslinking is performed at a temperature from about 40° C. to about 80° C.

7. The method of claim 1 wherein the tissue comprises bovine pericardium.

8. The method of claim 1 wherein the tissue comprises porcine pericardium.

9. The method of claim 1 wherein the tissue comprises mammalian pericardium

10. The method of claim 1 wherein the tissue comprises a porcine heart valve.

11. The method of claim 1 wherein the tissue is contacting a form within a bag under vacuum along with crosslinking agent.

12. The method of claim 1 wherein the tissue is contacting a porous form.

13. The method of claim 10 wherein the tissue is held on the porous form with suction with immersion of the tissue in crosslinking solution.

14. A bioprosthesis comprising tissue crosslinked using the method of claim 1.

15. A method for forming structures comprising fixed tissue, the method comprising:
placing a portion of tissue on a form with a contoured shape;
supporting the tissue on the form using pressure differentials; and
crosslinking the tissue supported on the form using a crosslinking solution.

16. The method of claim 15 wherein the form has the shape of a heart valve or a section of blood vessel.

17. The method of claim 16 wherein the form is produced from a three dimensional image of a natural structure, wherein the image is optionally corrected to remove defects.

18. The method of claim 15 wherein the form is porous.

19. The method of claim 15 wherein the pressure differential is created by placing the tissue, form and crosslinking solution into a bag that is evacuated and sealed.

20. The method of claim 15 wherein the form is porous and wherein the pressure differential is created by applying suction to a portion of the porous form away from the tissue while the tissue and form are immersed in crosslinking solution.

21. The method of claim 15 wherein the crosslinking solution comprises substantially unpolymerized glutaraldehyde.

22. A bioprosthesis comprising tissue crosslinked using the method of claim 15.

23. A method for forming structures comprising fixed tissue, the method comprising:
supporting a portion of tissue on a form with a contoured shape, wherein the form is porous; and
crosslinking the tissue in contact with the form using a crosslinking solution.

24. The method of claim 23 wherein the form has a shape corresponding to a heart valve or a portion of a blood vessel.

25. The method of claim 23 wherein the form is made by three dimensional printing.

26. The method of claim 25 wherein the printing is guided by an image of a corresponding biological structure, wherein the image is optionally corrected to remove defects in the biological structure.

27. The method of claim 26 wherein image is obtaining using a non-invasive medical imaging process of the patient who is the intended recipient of the prosthesis.

28. The method of claim 23 wherein the crosslinking solution comprises substantially unpolymerized glutaraldehyde.

29. A method of making a form useful for crosslinking tissue for a bioprosthesis intended for a specific patient, the method comprising:
imaging a defective tissue section in the specific patient;
adjusting the image to correct the defect and to form an image of a corrected tissue section; and
performing three dimensional printing of the image of the corrected tissue section.

30. The method of claim 29 wherein the imaging comprises obtaining an image with CT imaging, ultrasound imaging, MRI imaging or a combination.

31. The method of claim 29 wherein the defective tissue section is a heart valve or a portion of a blood vessel.

32. The method of claim 29 wherein the adjusting of the image comprises:
comparing the image of the defective tissue section with an idealized image of a healthy tissue portion;
identifying the defect and a corresponding section on the healthy tissue portion; and
mapping the corresponding section on the healthy tissue portion following scaling of the corresponding section to the defective tissue section, to replace the defective tissue section in the image with the scaled corresponding section.

33. A method for forming a bioprosthesis, the method comprising:
crosslinking a tissue portion in contact with a form made using the method of claim 29.
